(19) United States
(12) Patent Application Publication
(10) Pub. No.: US 2008/0114308 A1
(43) Pub. Date: May 15, 2008

(54) Vascular Access Port with Catheter Connector

(76) Inventors: Giorgio di Palma, Queensbury, NY (US); James J. Mitchell, Ballston Spa, NY (US); William C. Hamilton, Queensbury, NY (US)

Correspondence Address: ANGIODYNAMICS, INC., 603 QUEENSBURY AVENUE, QUEENSBURY, NY, 12804, US

(21) Appl. No.: 11/938,366

(22) Filed: Nov. 12, 2007

Related U.S. Application Data
(60) Provisional application No. 60/865,533, filed on Nov. 13, 2006.

(51) Int. Cl. A61M 5/00; A61M 31/00; A61M 39/10; A61M 37/00; A61M 39/00; A61M 25/16; A61M 25/18

(52) U.S. Cl. 604/246; 604/288.01; 604/533

(57) ABSTRACT

An implantable vascular access port assembly that has a septum, a reservoir, and a catheter connector assembly for use in connecting a catheter with the vascular access port. The catheter connector assembly has an O-ring, an O-ring seal, a locking ring, and a catheter connection plug, which components are located in secured adjacent relationship to one another, respectively, in the vascular access port. The catheter connector assembly securely locks the catheter inside of the vascular access port without compromising the lumen cross-sectional area and can withstand high pressures.

### CROSS-REFERENCE TO RELATED APPLICATIONS

This application claims priority to and the benefit of U.S. Provisional Application No. 60/865,533, filed on Nov. 13, 2006, which application is incorporated in its entirety in this document by reference.

### FIELD OF THE INVENTION

The present invention relates to the field of implanted vascular access devices. Particularly, the present invention pertains to the field of vascular access ports. More particularly, the present invention pertains to catheter connection devices for use with vascular access ports.

### BACKGROUND OF THE INVENTION

Implanted vascular access devices provide venous access to the central circulatory system of a patient. Vascular access ports are vascular access devices that are implanted into a patient's vascular system for applications in which repeated access to a patient's vascular system is necessary. Repeated access could be for the purpose of infusion of medications, prolonged intravenous feeding of fluids such as drugs or other fluids, parenteral nutrition solutions, blood products, imaging solutions, chemotherapy treatments, intensive antibiotic treatment, withdrawal of blood samples, or extracorporeal blood treatment protocols, such as hemodialysis, hemofiltration, or apheresis. Vascular access ports were developed to overcome problems associated with limited peripheral access in patients and to address the need for frequent venipuncture in patients with long-term venous therapies.

Typically, a vascular access port has a reservoir, an inlet septum in the center of the reservoir, and an outlet catheter that is placed into a vein in a patient. To implant the vascular access port in a patient, the distal tip of the catheter is introduced through an incision site in a patient, and the port is placed underneath the patient's skin, preferably in an area that provides good stability and does not interfere with patient mobility or create any additional pressure. To ensure ease of locating and inserting a needle into the septum, and to deter tissue erosion, it is important that the amount of skin over the port septum not be too thick or too thin. A tissue thickness over the septum of approximately 0.5 cm to approximately 2 cm is preferred. Typically, the catheter is placed into the superior vena cava through the subclavian, jugular, or cephalic vein. A subcutaneous pocket is created in the chest wall. The catheter is tunneled to the pocket. The catheter is connected to the reservoir of the port, placed in the pocket, and flushed with heparin solution. The implanted port is then sutured to the underlying fascia, and the incision is closed.

The port septum may then be percutaneously accessed through the patient's skin by using a needle, such as a non-coring needle, sometimes referred to as a Huber-style needle. The needle is penetrated through the patient's skin that overlays the access port and is inserted further through the septum and into the reservoir. The needle is used to deliver fluids, such as medications and drugs, into the port. The fluids exit through the reservoir and into the catheter, allowing treatment to be given directly into the bloodstream. Blood samples can also be taken in this way if needed.

Implanted vascular access ports may remain in a patient's body for extended periods of time. Thus, it is critical that the connection between the catheter and the vascular access port remains secure such that it does not leak or break while in the patient's body. Otherwise, patient treatment could be compromised. Additionally, the catheter connection needs to be able to withstand tensile and twisting pressures or shifts in weight by the patient that may loosen or break the catheter connection.

The process of connecting the catheter to the reservoir of the vascular access port may be difficult for a practitioner because the practitioner's gloves, as well as the vascular access port, may be wet and slippery inside of the patient's body as a result of contact with bodily fluids or other solutions.

Furthermore, if the catheter and the port need to be separated so that the catheter or port can be removed or exchanged, it can be difficult to manually disconnect the catheter from the port in such an environment. It is important that the connection and disconnection between the catheter and the port be able to be accomplished without additional surgery time, disruption, or trauma to the patient.

To minimize the difficulties associated with the connection and disconnection of the catheter and to help ensure the efficacy and safety of patient treatment, it is important that these connection and disconnection processes be as smooth, quick, and reliable as possible. The longer it takes the practitioner to connect or disconnect the catheter to the vascular access port, the greater the chance that effective treatment of the patient might be compromised. Thus, it is beneficial for the physician to be able to quickly connect and disconnect the catheter to and from the vascular access port with minimal insertion force and finger movement. This helps to keep surgery time to a minimum and helps to avoid any additional potential trauma to the patient.

Once a catheter is connected to a vascular access port, it is important that the catheter remains securely connected in order to prevent spontaneous disconnection or dislodgement of the catheter. Otherwise, the function of the catheter and the vascular access port can be impaired, causing an increase in inflammatory and thrombotic complications or extravasal administration of drugs. Dislocation or disconnection of the catheter from the port may also require the port to be removed in some cases, which can increase procedure time and cause unwanted complications.

To address these problems, several different types of vascular access port catheter connections have been proposed. Many of these connections require alignment and connection of a male port stem with a catheter lumen. Such alignment can be difficult to accomplish in a wet or slippery environment while wearing gloves. It can also be difficult to connect the catheter to the vascular access port stem because both the stem and the catheter lumen may be very small. Additionally, many stems have barbs of various sizes around their outer circumference. The barbs may act as an additional barrier over which the lumen of a catheter may have to be advanced, which might also require additional manual insertion force by the practitioner. When a catheter lumen is advanced over a barb, the catheter may become distressed and expand and/or change shape to fit the vascular access port stem. This may cause an increased risk of catheter deformation, dislodgement, leaking, or breaking.

In addition to properly positioning the catheter onto or within a vascular access port, some of the catheter connector devices in the prior art also may require a catheter lock to be attached around the outside surface of the catheter and manually adjusted on the catheter shaft before or after the catheter is inserted into the vascular access port stem, while making sure that the catheter remains straight and does not kink prior to securing the catheter lock. Otherwise, the catheter may break. The additional catheter lock may also be small and hard to handle for the practitioner. During the connection of the catheter with the vascular access port, if the catheter and lock are connected and then disconnected, the end of the catheter may need to be re-trimmed to ensure a secure connection between the catheter and the vascular access port, thereby increasing the procedure time. This additional step can be time-consuming and labor intensive for the practitioner, especially in a wet, slippery environment, and could potentially cause unnecessary trauma to the patient.

Finally, in some proposed catheter connections, there is a possibility that the connection between the catheter and the vascular access port may require such a great amount of manual force to insert the catheter into the vascular access port that the port could be dislodged from its pocket, or other unwanted trauma could occur inside the patient's body that could compromise the efficacy of patient treatment. If the catheter connector is large or cumbersome, the catheter connector might also be too difficult to work with or might unnecessarily increase surgery time or potentially cause other complications.

Current vascular access port catheter connections do not provide a catheter connector device that allows a practitioner to manually insert the catheter into a vascular access port with a minimal amount of insertion force, where it can be locked inside of the port using a locking ring mechanism. Neither do current vascular access port catheter connections provide a catheter connector that can be removed from the port with minimal force and finger movement, after being inserted into the port, by manually pressing a catheter connection release, thereby allowing the catheter to easily be released from the port.

There has been, and continues to be, a need for a solution to the above mentioned problems, such as a vascular access port catheter connector that is easy to use, requires minimal manual insertion and disconnection force, and provides a quick, safe, and secure, yet controllable releasable connection between the catheter and the vascular access port.

A solution to the above-mentioned problems would be a vascular access port with a catheter connector assembly that allows the catheter to be securely locked inside of the vascular access port, after the catheter is manually inserted into the port, with minimal force and finger movement in a surgical environment. Such a catheter connector assembly would also allow the catheter to be easily removed by a practitioner, if and when desired, by manually releasing the catheter connection device using a catheter connection release.

The present invention addresses the problems in the prior art by providing a catheter connector assembly that allows a practitioner to manually insert a catheter into a vascular access port with minimal force and finger movement in a small environment that can be wet and slippery. The catheter connector assembly of the present invention also ensures that the catheter will securely remain inside of the vascular access port during treatment and will not become disconnected unless and until desired.

Accordingly, it is a purpose of the present invention to provide a vascular access port with a catheter connector assembly that allows a practitioner to connect a catheter to a vascular access device, such as a vascular access port, with minimal time, insertion force, and finger movement.

Another purpose of the present invention is to provide a vascular access port with a catheter connector assembly that does not require any additional pieces or steps.

Another purpose of the present invention is to provide a catheter connector assembly which allows for a secure connection between the catheter and the vascular access port that can withstand additional outside pressures while implanted inside of a patient body.

Another purpose of the present invention is to provide a catheter connector assembly which can withstand high pressures from within the vascular access port, such as occur with CT injections.

Another purpose of the present invention is to provide a catheter connection release which allows a practitioner to manually release the catheter from the vascular access port with minimal time, force, and finger movement, if desired, or if the catheter or port needs to be removed or exchanged.

In another aspect of the invention, a method of using the catheter connector assembly to connect a catheter and a vascular access port is presented.

Various other objectives and advantages of the present invention will become apparent to those skilled in the art as more detailed description is set forth below. Without limiting the scope of the invention, a brief summary of some of the claimed embodiments of the invention is set forth below. Additional details of the summarized embodiments of the invention and/or additional embodiments of the invention may be found in the Detailed Description of the Invention.

### BRIEF SUMMARY OF THE INVENTION

The present invention provides a catheter connector assembly for connecting a catheter to a vascular access port. The catheter connector assembly of the present invention is advantageous in view of current vascular access port catheter connections because it allows a catheter to be connected to a vascular access port more easily, quickly, and reliably, with less insertion force compared to the prior art, and without any additional steps or parts.

The catheter connector assembly has a catheter and a vascular access port with a port bore that is adapted to house the catheter connection assembly within a portion of the port bore. The assembly also has a channel that is in fluid communication with the port bore and a reservoir, and a locking ring that has finger members that extend inwardly with inner surfaces that define a locking ring lumen. When a portion of the outer wall of the catheter is positioned within the locking ring lumen in frictional contact with the finger members, the finger members flex from a first, unbiased, position to a second, biased, position.

The catheter connector assembly also has an O-ring, an O-ring seal and a catheter connection plug, all mounted just inside the opening of a vascular access port to help connect the catheter to the port. The catheter connector assembly helps to ensure that the catheter will securely remain in the port by providing an interference fit around the outer surface of the catheter and allows a catheter to be inserted into a vascular access port with minimal force or resistance and finger movement. The catheter connector assembly may also have a catheter connection release mechanism coaxially arranged around the catheter shaft and partially extending within the port bore. The catheter connector release may also have a forward-facing front surface that is mounted outside of the vascular access port that allows a practitioner to manually disengage the catheter from the port when the outer face of the catheter release mechanism is depressed. The instant invention also encompasses a method of inserting a catheter into a vascular access port that includes positioning a locking ring within the port bore and inserting the outer wall of the catheter into the locking ring lumen, thereby causing the finger members of the locking ring to flex, and removing the catheter by axially moving a catheter release mechanism.

The catheter connector assembly of the present invention decreases the chance that a catheter or port will dislodge or leak during and after the connection of the catheter to a port because the catheter connector assembly allows a catheter to be securely connected to the vascular access port while implanted in the patient body. The catheter connector assembly also allows a catheter to be easily, quickly, and reliably removed compared to the prior art, using a catheter connection release device.

### BRIEF DESCRIPTION OF THE SEVERAL VIEWS OF THE DRAWINGS

These and other features of the preferred embodiments of the invention will become more apparent in the detailed description in which reference is made to the appended drawings wherein:

FIG. 1A is a perspective view of the vascular access port assembly with a single lumen catheter inserted into the vascular access port opening, in accordance with the present invention.

FIG. 1B is a perspective view of the vascular access port assembly without a catheter inserted into the vascular access port opening, in accordance with the present invention.

FIG. 2A a side view of the vascular access port of FIG. 1, in accordance with the present invention.

FIG. 2B is a cross-sectional view of the vascular access port of FIG. 2A, in accordance with the present invention.

FIGS. 3A and 3B are partial enlarged cross-sectional views of the catheter connector assembly housed within a portion of the port bore of the vascular access port and the port bore, respectively, in accordance with the present invention.

FIG. 4 is a partial exploded view of the vascular access port including the vascular access port assembly, the catheter connector assembly and the catheter, in accordance with the present invention.

FIG. 5A is a plan view of the O-ring of the catheter connector assembly, in accordance with the present invention.

FIG. 5B is a cross-sectional view of the O-ring of FIG. 5A taken along line A-A.

FIG. 6A is a plan view of the O-ring seal of the catheter connector assembly, in accordance with the present invention.

FIG. 6B is a cross-sectional view of the O-ring seal of FIG. 6A taken along line A-A.

FIG. 7A is a plan view of the locking ring of the catheter connector assembly in accordance with the present invention.

FIG. 7B is a side view of the locking ring of FIG. 7A.

FIGS. 8A and 8B are a plan view and a side cross-sectional view, respectively, of the connection plug of the catheter connector assembly, in accordance with the present invention.

FIG. 9A is a plan view of the assembled O-ring seal, locking ring, and connection plug of the catheter connector assembly in accordance with the present invention.

FIG. 9B is a cross-sectional view of the assembly of FIG. 9A taken along line A-A.

FIG. 10 is a partial exploded view of an additional embodiment of the vascular access port assembly of the present invention including the catheter connector release element, in accordance with the present invention.

FIG. 11A is plan end view and FIG. 11B is a side view of the catheter connector release element of the embodiment illustrated in FIG. 10, in accordance with the present invention.

### DETAILED DESCRIPTION OF THE INVENTION

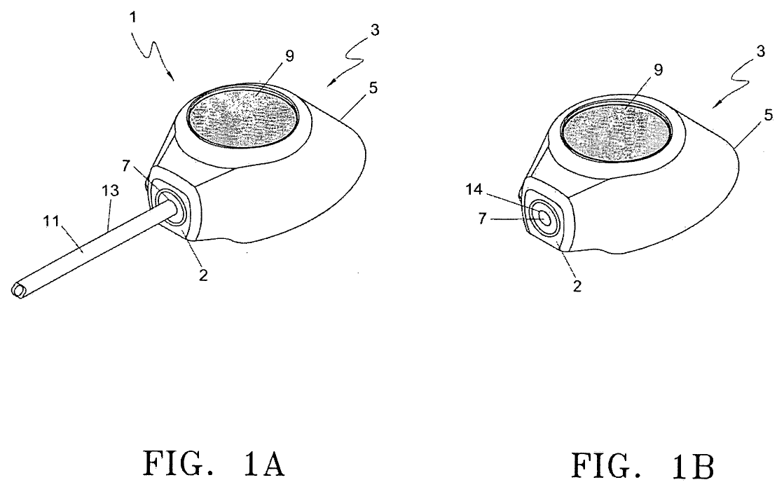

The following detailed description should be read with reference to the drawings, in which like elements in different drawings are identically numbered. The drawings, which are not necessarily to scale, depict selected preferred embodiments and are not intended to limit the scope of the invention. The detailed description illustrates by way of example, not by way of limitation, the principles of the invention. The present invention is illustrated in FIGS. 1 through 11.

The present invention is more particularly described in the following exemplary embodiments that are intended to be illustrative only since numerous modifications and variations therein will be apparent to those skilled in the art. As used herein, “a,” “an,” or “the” can mean one or more, depending upon the context in which it is used. The preferred embodiments are now described with reference to the figures, in which like reference characters indicate like parts throughout the several views.

Ranges may be expressed herein as from “about” one particular value, and/or to “about” another particular value. When such a range is expressed, another embodiment includes from the one particular value and/or to the other particular value. Similarly, when values are expressed as approximations by use of the antecedent “about,” it will be understood that the particular value forms another embodiment.

Referring to FIGS. 1A-B and 2A-B, catheter connector assembly 15 of the present invention allows a practitioner to quickly and reliably insert a catheter 11 into a vascular access port 3 with a minimal amount of force and finger movement. The catheter connector assembly 15 inside of the vascular access port 3 of the present invention connects the catheter 11 to port housing 5 and helps to retain the catheter 11 in position in the vascular access port 3.

The vascular access port assembly 1 of the present invention, illustrated in FIG. 1A, has a vascular access port 3 with a septum 9. The vascular access port septum 9 is surrounded by vascular access port housing 5. The housing 5 may be composed of any suitable material. Preferably, the housing 5 is composed of a hard plastic or engineering resin such as polysulfone or acetal. Titanium or other metal may also be used. The septum 9 of the present invention may be composed of any suitable material. Preferably, the septum 9 is composed of silicone. The septum 9 may have a diameter of approximately between 9 and 13 mm. Preferably, the septum 9 has a diameter of approximately 11 mm.

The vascular access port assembly 1 is illustrated with a catheter shaft 11 having an outer wall 13 inserted into the port opening 7 of the vascular access port 3. The port opening 7 of the vascular access port 3 is approximately 0.095 inches in diameter and has an inner wall 14. After the catheter shaft 11 is inserted into the vascular access port 3 through port opening 7, the end of the catheter shaft 11 is advanced through the catheter connector assembly 15, where the catheter shaft 11 is secured inside of the vascular access port 3.

The catheter 11 of the present invention may have a useable length of approximately 30 inches, an outer diameter of approximately 0.095 inches, an inner diameter of approximately 0.056 inches, and a wall thickness of approximately 0.018 inches. Although a 7 French catheter is used in the present invention, a catheter that is from 4 French to 14 French may also be used. The dimensions of the vascular access port assembly 1, including the vascular access port 3 and the catheter connector assembly 15, may also vary in size relative to one another and relative to the catheter 11, depending on what size catheter 11 is to be inserted into the port 3.

The catheter connector assembly 15 can be used with any type of vascular access port 3. The catheter 11 is preferably made of any suitable soft material, such as, but not limited to any polyurethane type material. The catheter 11 of the present invention may also be a single or double lumen catheter. The catheter 11 or port 3 may also have a radiopaque marker(s) anywhere throughout the catheter 11 or port 3 for further visibility of the catheter 11 or port 3 in the patient's body. The catheter 11 may also have depth markings every centimeter throughout the catheter 11. The type of catheter 11 and lumen size that is chosen depends on the patient's needs.

The catheter 11 of the present invention may be pre-attached to or pre-assembled with the vascular access port 3, or it may be manually attached to the vascular access port 3 by a practitioner during the placement procedure. If the catheter 11 is separately attached to the port 3 by the practitioner, as illustrated in FIGS. 1 through 9 in the preferred embodiment, the catheter 11 may not be removed after it is attached, unless the catheter release mechanism 69 used to disconnect the catheter 11 from the vascular access port 3, as described below. This provides a secure and leak-proof connection between the catheter 11 and the port 3. The vascular access port assembly 1 of the present invention may be MRI compatible, and the secure catheter connection allows the port 3 to withstand high pressures, such as occur during CT injections.

The components of the catheter connector assembly 15 enable the catheter 11 to be securely inserted into and maintained inside of the vascular access port 3. The catheter connection assembly 15 of the present invention thereby provides a reliable connection between the catheter 11 and the port 3 and requires minimal force to insert the catheter 11 into the vascular access port 3, due to the catheter connection assembly 15, which component parts allow for a tight and secure fit between the catheter 11 and the port 3.

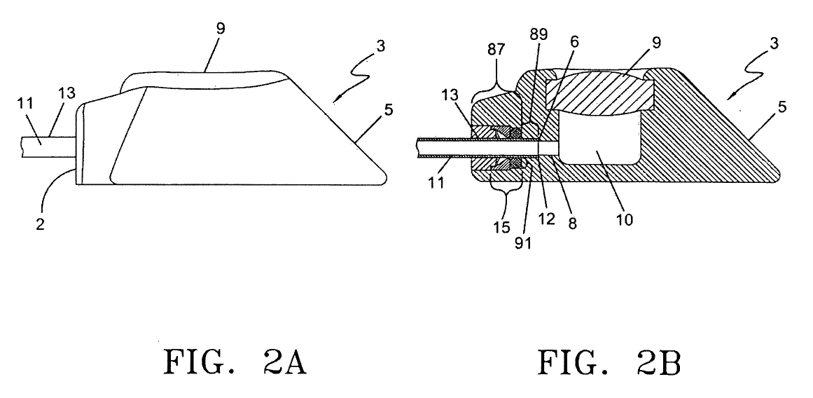

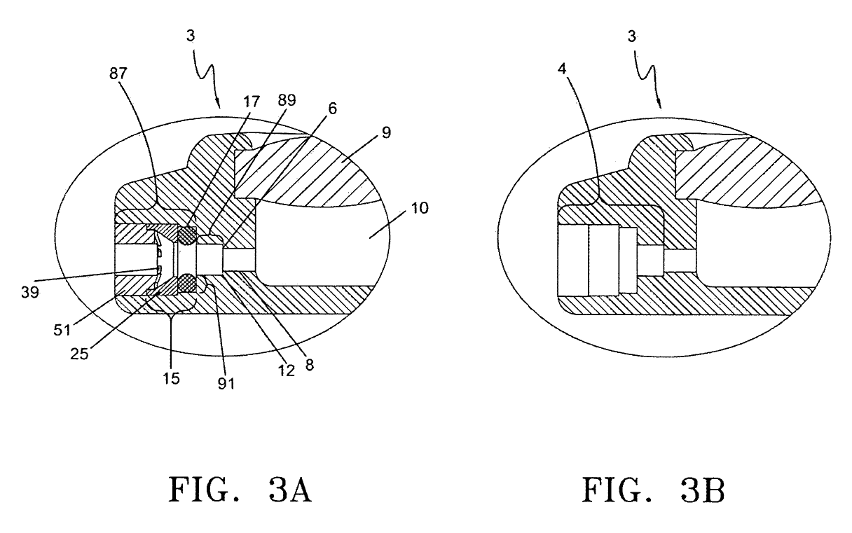

FIG. 1B illustrates the vascular access port assembly 1 of FIG. 1A, including the vascular access port 3, septum 9, housing 5, and port opening 7 with inner wall 14, without the catheter 11 inserted into the port opening 7 of the vascular access port 3. The catheter connector assembly 15, also illustrated in FIGS. 2B and 3A, is located inside portions of the port bore 4 of the vascular access port 3. The advantage of having the catheter connector assembly 15 located just inside of the vascular access port 3 is that it allows the vascular access port 3 to be contained in a smaller area within a patient body, compared to vascular access ports with a stem extending externally from the port housing 5. This also allows the vascular access port 3 to be less intrusive when placed inside of a patient. Additionally, the port 3 of the present invention eliminates the need to align a port stem with a catheter, and instead, the face or sidewall 2 of the vascular access port 3 may be generally aligned with the catheter 11 so that the catheter 11 can be inserted into the port bore 4, which requires less precision.

The port 3 may be transparent to enable the practitioner to visually observe when the catheter 11 has been completely inserted through opening 7 and into the port bore 4 of the vascular access port 3. The insertion of the catheter 11 into the vascular access port 3 can thus be verified via both visual and tactile indications. Although the preferred embodiment of the vascular access port 3 has no stem, the vascular access port 3 may also have a stem that extends outside of and beyond the catheter port 3. The stem may also be transparent to enable a visible indication of complete insertion of the catheter shaft 11. The vascular access port 3 of the present invention may also be of a dual port design, or it may have one port. The vascular access port 3 may also have a dual durometer septum. The vascular access port 3 may also be made of titanium, stainless steel, or molded plastic.

FIG. 2A illustrates a side view of the vascular access port assembly 1 of FIG. 1A, with the catheter shaft 11 extending from face or sidewall 2 of the port housing 5. In FIG. 2B, a cross-sectional view of the preferred embodiment of the vascular access port assembly 1 of FIG. 2A is illustrated. The vascular access port assembly 1 has a vascular access port 3 comprised of a housing 5, in which a septum 9 and a reservoir 10 are located. Reservoir 10 is in fluid communication with catheter shaft 11 through channel 8. Fluid injected using a needle placed through septum 9 into reservoir 10 flows into the catheter through channel 8. The proximal end 12 of the catheter 11 is positioned in the port 3 with catheter end 12 in contact with and abutting the outer edge 6 of channel 8. Coaxially surrounding the catheter shaft 11 within the port housing 5 is catheter connector assembly 15.

The catheter connector assembly 15 has several components, which are illustrated as assembled in the enlarged view of FIG. 3A. The catheter 11 has been removed from FIGS. 3A and 3B for clarity of the catheter connection assembly 15. The catheter connector assembly 15 has an O-ring 17, an O-ring seal 25, a locking ring 39, and a connection plug 51, respectively, located adjacent to and abutting against each other in the assembled state inside of the port bore 4 of the vascular access port 3. All catheter connector assembly 15 components form a common lumen extending substantially co-axial to the longitudinal axis of the port bore 4 through which the catheter shaft 11 is inserted. O-ring 17 is located at the inner most position within port bore 4 and functions to provide a seal to prevent fluid loss within the fluid pathway. Abutting up against the O-ring 17 is O-ring seal; 25 which stabilizes the position of O-ring 17 and provides a contoured cavity within which locking ring 39 is held, as will be explained in greater detail below. Connection plug 51 is located at the outer most position within the port bore 4 and can be aligned such that the outer surface of the connection plug 51 is flush with the face or sidewall 2 of port housing 5. In one aspect, the connection plug 51 retains the locking ring 39 in position and provides a barrier to prevent locking ring 39 from flexing in a radially outward direction.

As illustrated in FIGS. 3A and 3B, in one aspect, the port bore 4 has a first portion 87 that extends inwardly from a face or sidewall 2 of the vascular access port 3 to shoulder surface 91 and a second portion 89 that extends inwardly from the shoulder portion 91 and terminates at an edge 6 of channel 8. The first portion 87 is adapted to house the catheter assembly 15. In a particular aspect, the first portion 87 can have sub-sections with varying diameters. For example, the first portion 87 can have a proximal section with a diameter that is greater than a distal section of the first portion 87. In a further aspect, the first portion 87 of the port bore 4 has a diameter that is greater than the diameter of the second portion 89. In yet a further aspect, the second portion 89 of the port bore 4 can have a diameter substantially the same as the outer diameter of the catheter 11 such that a portion of the distal end 12 of the catheter can be inserted into the second portion 89 as selectively desired, so as to allow for a suitable interference fit. The channel 8 of the vascular access port 5 can have a diameter that is less than the outer diameter of the catheter 11. According to another aspect, the channel 8 can have a diameter that is substantially equal to an inner diameter of the catheter 11, as shown in FIG. 2B. FIG. 3B illustrates the port bore 4 without the catheter connector assembly 15 inside of the port bore 4.

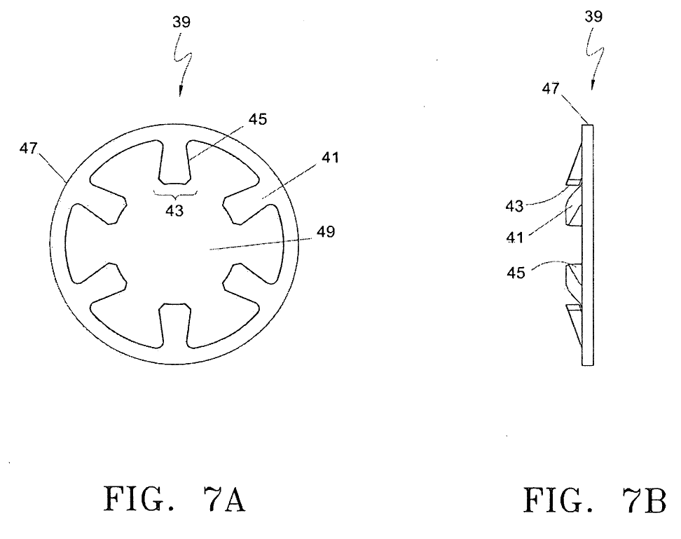

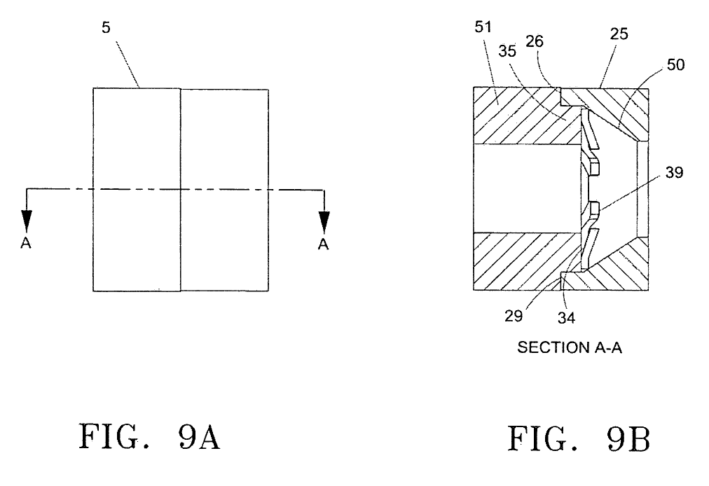

To connect the catheter shaft 11 to the port 3, the catheter end 12, shown in FIG. 2B, is inserted into the port bore 4 and through the connection plug 51 lumen defined therein. As the catheter shaft 11 is advanced through the lumen of the locking ring 39, the finger members 41 (shown in FIGS. 7A and 7B), flex inwardly toward the port reservoir 10 and outwardly away from the longitudinal axis of the port bore 4. In one aspect, the shaft 11 is then advanced through the O-ring seal 25 and O-ring 17 until the catheter end 12 abuts up against the edge 6 of channel 8. If the catheter shaft 11 is pulled in the opposite direction, the fingers 41 will begin to flex in the opposite direction, but will be prevented from further flexing by the inner back face 34 (illustrated in FIG. 9B) of the connection plug 51. Once inserted fully, the catheter shaft 11 is held in a stationary position by the finger members 41 which are restricted from flexing outwardly by the connection plug 51. Thus, the catheter connection assembly 15 allows insertion of a catheter shaft 11, but prevents removal once secured in place.

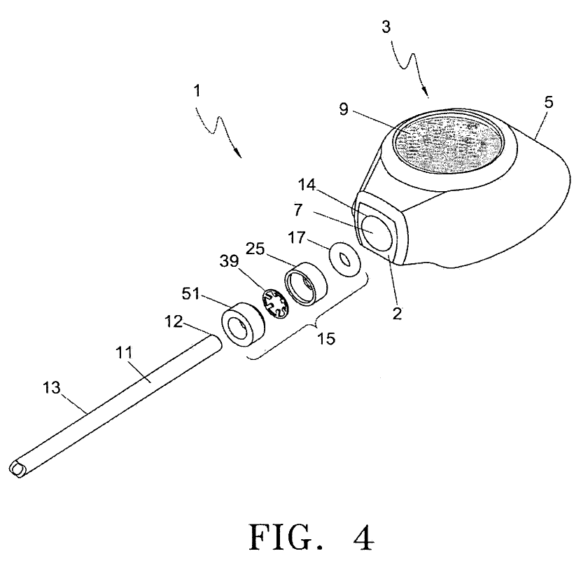

The preferred embodiment of the present invention is also illustrated in FIG. 4, which is a partially exploded view of the vascular access port assembly 1. The vascular access port assembly 1 includes the assembled vascular access port 3, the components of the catheter connection assembly 15, and the catheter shaft 11. The vascular access port 3 has a vascular access port bore 4 and a septum 9 inside of the port 3, which is surrounded by housing 5. The components of the catheter connector assembly 15 are illustrated, which include O-ring 17, O-ring seal 25, locking ring 39, and connection plug 51. In the assembled state, the catheter connection assembly 15 components are located adjacent to and abutting up against one another.

The end 12 of the catheter shaft 11 is inserted into and through the components of the catheter connector assembly 15, which are located just inside of the vascular access port bore 4, in the assembled state. As the catheter shaft 11 is inserted through the catheter connector assembly 15, the O-ring 17, O-ring seal 25, and connection plug 51, the locking ring 39 of the catheter connector assembly 15 locks down around the outer wall 13 of the catheter shaft 11, thereby securing the catheter shaft 11. The lock-down of the locking ring 39 around the catheter shaft 11 also prevents the catheter shaft 11 from being removed from the vascular access port 3.

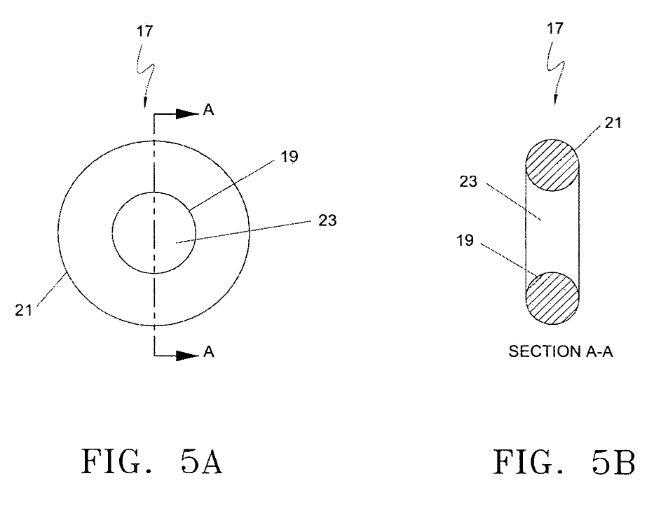

The O-ring 17 of the catheter connector assembly 15 is illustrated in FIG. 5A. The O-ring 17 is located inside of the vascular access port assembly 1 closest to the channel 8 of the vascular access port 3. In one aspect, the O-ring 17 is configured to be seated against the shoulder surface 91 (illustrated in FIG. 3A) of the first portion 87 of the port bore 4 and defines an O-ring lumen 23 that has a diameter that is less than the outer diameter of the catheter 11. As the distal end 12 of the catheter 11 is inserted into the catheter connector assembly 15, the inner surface 19 of the O-ring 17 becomes disposed around the outer wall 13 of the distal end 12 of the catheter shaft 11 and forms an interference fit between the inner surface 19 of the O-ring 17 and the outer wall 13 of the catheter shaft 11. The O-ring 17 helps to hold the catheter 11 in place in relation to the vascular access port 3. The O-ring 17 seals around the outer diameter of the catheter 11 and allows the entire vascular access port assembly 1 to hold pressure without leaking.

In the assembled state, the O-ring 17 of the catheter connector assembly 15 is sealed inside of an O-ring seal 25 and forms an interference fit between the O-ring 17 and the O-ring seal 25. The O-ring 17 may be composed of any suitable material. More particularly, the O-ring 17 may be composed of silicone. The cross-sectional width of the O-ring is approximately 0.070 inches. The O-ring also has a lumen 23, an inner diameter of approximately 0.088 inches and an outer diameter of approximately 0.228 inches. Thus, in one example and not meant to be limiting, the O-ring 17 thus provides an interference fit of approximately 0.002 inches per side with catheter shaft 11 which has an outer diameter of 0.092 inches, without compromising the luminal diameter of the shaft 11.

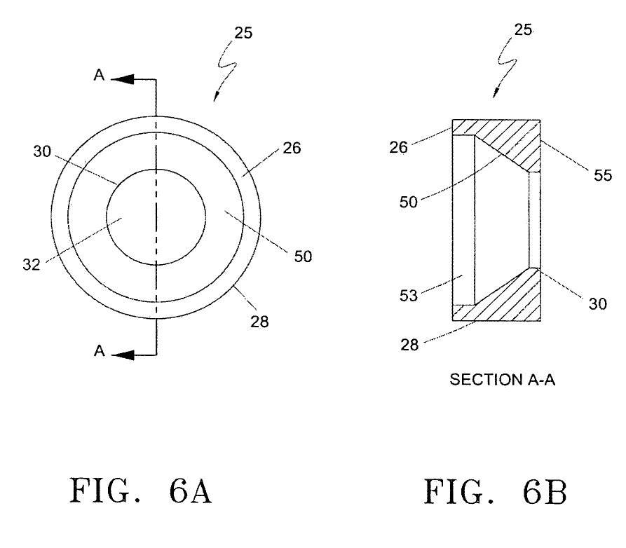

The O-ring seal 25 is illustrated in FIGS. 6A and 6B. The O-ring seal functions to hold the O-ring in place inside of the catheter connector assembly 15 around the catheter shaft 11. In addition to retaining the O-ring 17 within the catheter connector assembly 15, the O-ring seal 25 also helps to retain the locking ring 39 in place within the catheter connector assembly 15 and around the outer wall 13 of the catheter 11. As shown in the end view of FIG. 6A, the O-ring seal 25 has an outer wall 28, with a front facing outer surface 26 and an inwardly facing outer surface 55. O-ring seal 25 also has a tapered inner surface 50 terminating at inner most wall segment 30 which defines O-ring seal lumen 32. In one exemplary aspect, the diameter of the outer wall 28 of the O-ring seal 25 is substantially the same as the diameter of the first portion 87 of the port bore 4 and is configured to be received therein a portion of the first portion 87 of the port bore 4. In another aspect, the inwardly facing outer surface 55 abuts the O-ring 17 such that the O-ring lumen 23 and the O-ring seal lumen 32 are substantially co-axial.

The O-ring seal 25 functions to retain O-ring 17 in position and to house the locking ring 39. In the current embodiment, the O-ring seal 25 has an outer diameter of approximately 0.268 inches. The O-ring seal 25 has a length between the front facing outer surface 26 and inwardly facing outer surface 55 of approximately 0.085 inches. The O-ring seal 25 also has an inner diameter of approximately 0.230 inches at the front facing outer surface 26 and a diameter of approximately 0.125 inches at the inwardly facing outer surface 55. The tapered inner surface 50 has an angle of approximately 55 degrees from the front facing outer surface 26 to the inwardly facing outer surface 55. The O-ring seal 25 may be composed of any suitable material. Preferably, the O-ring seal 25 may be composed of a hard plastic or engineering resin such as polysulfone or acetal.

Referring back to FIG. 3A, O-ring seal 25 contacts and holds O-ring 17 in a slightly compressed position when assembled. Specifically, the inwardly facing outer surface 55 of O-ring seal 25 abuts up against the outer portion of O-ring 17 wall. O-ring seal lumen 32 is of a reduced diameter at inwardly facing surface 55 relative to the opening at front facing outer surface 26 to ensure that the O-ring lumen 23 and the O-ring seal lumen 32 are aligned and that the compression forces are equally dispersed along the O-ring 17. The O-ring seal lumen 32 also houses the locking ring 39. In one aspect, the locking ring 39 is mounted therein a portion of the tapered inner surface 50 of the O-ring seal 25. In another aspect the locking ring 39 can be positioned within the O-ring seal lumen 32 at its widest diameter, in a position adjacent to the beginning of the tapered inner surface 50, as shown in FIG. 3A. The locking ring 39, which will be described in more detail below, is held in place on one side by the tapering inner surface 50 of the O-ring seal 25 and on the other side by the connection plug 51.

A front view and side view of the locking ring 39 component is illustrated in FIGS. 7A and 7B. The locking ring 39 is positioned within the O-ring seal 25 and is in contact with the catheter connection plug 51. The locking ring 39 has a circumferential edge 47, a lumen 49, and locking ring fingers 41 which extend from the circumferential edge 47 of the locking ring 39 inwardly into the lumen 49 of the locking ring 39. Each locking ring finger member 41 of the locking ring 39 is defined by an inner surface 43 and a side surface 45. The inner surfaces 43 of the locking ring finger members 41 contact and hold the outer wall 13 of the catheter shaft in place when assembled. The finger members 41 in the embodiment shown have tapered edges and a smooth, non-sharp inner surface 43 so as not to damage the catheter shaft 11 when assembled. The length between the outer most point of the outer wall 47 of the locking ring 39 and the outer face 43 of the locking ring is approximately 0.060 inches. The width of locking ring 39, shown in FIG. 7B, is approximately 0.050 inches.

In one aspect, the respective inner surfaces 43 of the plurality of finger members 41 define a locking ring lumen 49 having a first diameter that is less than the outer diameter of the catheter shaft 11 when the finger members 41 are in a first, unbiased position. In a further aspect, the locking ring lumen 49 has a second diameter that is greater than the first diameter and is less than or substantially equal to the outer diameter of the catheter 11 when the plurality of finger members 41 is in a second, biased position in which a portion of the outer wall 13 of the catheter is positioned therein the locking ring lumen 49 in frictional contact with the respective inner surfaces 43 of the plurality of finger members 41. In another aspect, the circumferential edge 47 of the locking ring 39 can be positioned in a locking ring plane that extends substantially transverse to a longitudinal axis of the port bore 4. In this aspect, in the first, unbiased position, the plurality of finger members 41 extends inwardly away from the a locking ring plane toward the edge 6 of the channel 8 at an acute angle relative to the longitudinal axis of the port bore 4.

Although the locking ring 39 illustrated in FIGS. 7A and 7B has six finger members 41, the locking ring 39 may have anywhere from at least two locking finger members 41 to as many locking finger members 41 as is physically possible, as long as the locking finger members 41 are capable of applying force around the outer wall 13 of the catheter shaft 11, such that the force exerted on the outer wall 13 of the catheter shaft 11 is equalized in order to keep the catheter 11 aligned in the center of the lumen 49 of the locking ring 39. The finger members 41 may also be of different shapes such as T-shaped with arched end flanges. In one exemplary aspect, the plurality of finger members 41 are angularly spaced from each other at substantially the same distance so that force exerted on the outer wall 13 of the catheter shaft 11 can be substantially equalized.

The locking ring 39 has an outer diameter of approximately 0.216 inches corresponding to the O-ring seal lumen 32 diameter at front facing outer surface 26 of O-ring seal 25. The locking ring 39 inner diameter, defined by a non-continuous circle connected by the outer surfaces 43 of the locking ring finger members 41, is approximately 0.071 inches. As noted above, this diameter increases after insertion of the catheter shaft 11 through the locking ring 39. When the catheter 11 is inserted through lumen 49, the inner diameter expands from approximately 0.071 inches to a flexed second inner diameter of approximately 0.092 inches.

As the catheter 11 is inserted into the port 3, through the catheter connector assembly 15, and into the locking ring lumen 49, the locking ring finger members 41 flex outwardly away from the circumferential edge 47 of the locking ring 39 and toward the O-ring 17 to accommodate the outer wall 13 of the catheter shaft 11. The flexed inner diameter of the locking ring finger members 41 increases to accommodate the outer diameter of catheter 11 as it is inserted into and through the lumen 49 of the locking ring 39. After the catheter 11 is inserted into and through the lumen 49, the locking ring finger members 41 of the locking ring 39 lock down on the outer wall 13 of the catheter shaft 11 and circumferentially surround the outer wall 13 of the catheter 11.

As the locking ring finger members 41 are flexed outwardly from the locking ring 39 toward the O-ring 17, the angle of the locking ring finger members 41 from the outer wall 47 of the locking ring 39 decreases. Once the catheter 11 is manually pushed all the way through the catheter connector assembly 15 until it can advance no further, the flexed locking ring finger members 41 radially contact and grab portions of the outer surface 13 of the catheter 11, thereby preventing the catheter 11 from being removed from the vascular access port 3.

The locking ring 39 is made of a malleable material with shape-memory characteristics, such as nitinol, to allow expansion from a static inner diameter to a flexed inner diameter when the catheter 11 is fully inserted into the catheter connector assembly 15. Nitinol is an alloy material that is well-suited for the locking ring 39 because of its shape-memory characteristics, which enables the locking ring finger members 41 to automatically return to a smaller inner diameter (ID) upon release from a flexed position. The locking ring 39 may also be made of other shape-memory materials, such as, but not limited to, stainless steel, titanium, or nickel-titanium alloys.

When the catheter 11 is inserted into the port 3, the catheter 11 stops up against port channel 8, which may be indicated by visual and/or tactile observation. The locking finger members 41 of the locking ring 39 are engaged against the outer wall 13 of the catheter shaft 11, such that the catheter shaft 11 may not be removed from the port 3. After the catheter shaft 11 abuts up against the channel 8 of the port 3, the locking ring finger members 41 come to rest against the outer wall 13 of the catheter shaft 11, the locking ring finger members 41 grab and retain the catheter 11 inside of the vascular access port 3, preventing the catheter 11 from being released. The locking ring finger members 41 may become slightly embedded in the outer wall 13 of the catheter shaft 11 after they come to rest against the outer wall 13 of the catheter 11. The locking ring finger members 41 contact the outer wall 13 of the catheter 11 at the inner surface 43 of the locking rings 41, which have a width of 0.020 inches. Thus, the outer wall 13 of the catheter shaft 11 is contacted for a total length of 0.120 inches at six equally spaced sections of 0.020 inches circumferentially around the outer wall 13 of the catheter shaft 11.

The outer wall 13 of the catheter 11 is only indented slightly at those areas where the locking ring finger members 41 press in on the outer wall 13 of the catheter shaft 11. The amount that the outer wall 13 of the catheter shaft 11 decreases when the locking ring finger members 41 make contact with the outer wall 13 is negligible and does not compromise the lumen cross sectional area of the catheter shaft 11. For instance, the outer wall 13 of the catheter 11 may be indented no more than from between about 0.001 and 0.002 inches to about 50% of the wall thickness of the outer wall 13 of the catheter shaft 11, compared to the original outer diameter of approximately 0.092 inches of the catheter shaft 11. More specifically, after the catheter 11 of the present invention, with an outer diameter of approximately 0.092 inches, has been inserted into a locking ring 39 with an inner diameter of approximately 0.071 inches, an interference fit of approximately 0.021 inches is achieved.

The amount of compression or shape change of the catheter shaft 11 depends upon the size and type of catheter 11 used, as well as the material used in the catheter 11. The variation in the wall thickness will thus vary depending on these factors. Even if the outer wall 13 of the catheter shaft 11 is indented slightly, the catheter 11 is capable of substantially returning to its original outer diameter of approximately 0.092 inches because of the elasticity of the outer wall 13 of the catheter 11.

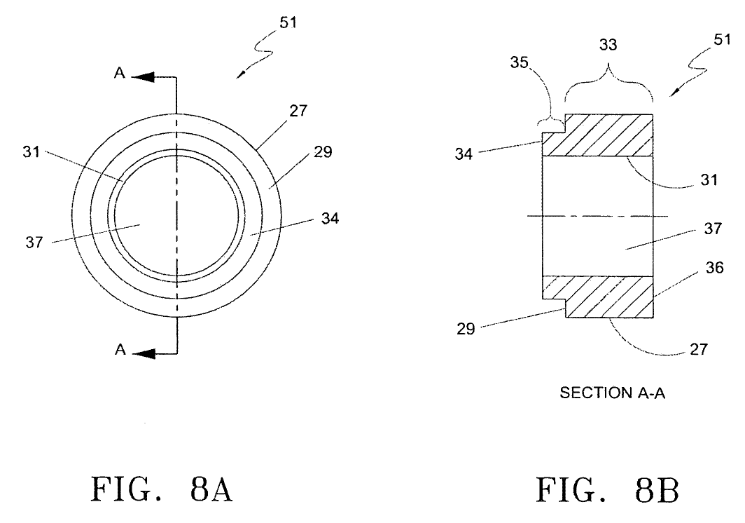

The catheter connection plug 51 is illustrated in FIGS. 8A and 8B. The catheter connection plug 51 has a primary portion 33 defined by outer wall 27 and a secondary portion 35 of reduced diameter. The connection plug 51 has a total length of approximately 0.175 inches, which is made up of the primary portion 33, which has a length of approximately 0.160 inches and the secondary portion 35, which has a length of approximately 0.015 inches. The wall thickness of outer back face 29 of the first portion 33 of the catheter connection plug 51 is approximately 0.051 inches. Lumen 37, defined by an inner wall 31, extends through the catheter connection plug 51. The catheter connection plug 51 has an inner diameter of approximately 0.095 inches and an outer diameter of approximately 0.279 inches. The secondary portion 33 of the connection plug 51 has an outer diameter of approximately 0.213 inches to allow insertion into the lumen 32 of O-ring seal 25 when assembled. The connection plug 51 may be composed of a hard plastic or engineering resin such as polysulfone or acetal, although any suitable material may be used. In one aspect, the diameter of the primary portion 33 of the connection plug 51 can be substantially the same as the diameter of the first portion 87 of the port bore 4 and is configured to be received therein a portion of the first portion 87 of the port bore 4.

When assembled, the flat outer front surface 36 of the catheter connection plug 51 is flush with the exterior surface of port housing 5. Although the connection plug 51 is depicted as having a flat surface, the connection plug 51 may also have an angled outer front surface 36 of approximately 45 degrees outwardly from the axis of the connection plug 51, to match the contour of the housing.

The inner back face 34 of outer connection plug 51 is designed to maintain the locking ring 39 fixed in position within the O-ring seal, as shown in FIG. 9B, which illustrates the sub-assembly of the locking ring 39, O-ring seal 25 and connection plug 51. The secondary portion 35 of the catheter connection plug 51 is inserted into the lumen 32 of the O-ring seal and advanced until inner back face 34 is prevented from further advancement by the tapered inner surface 50 of O-ring seal 25. The inner back face 34 also contacts the locking ring 39 maintaining it in place. More specifically, the inner back face 34 provides a barrier blocking the locking ring 39 finger members 41 from flexing outwardly toward the port bore 4 when the catheter shaft is being pulled. The finger members 41 will flex only until they contact inner back face 34 and then be stopped from flexing further by the inner back face surface 34 of O-ring seal 25.

The outer back face 29 of the first portion 33 of the catheter connection plug 51 functions to maintain the O-ring seal 25 in proper position. The O-ring seal 25 and the connection plug 51 may be welded together if desired using bonding glue, gel, or other adhesive. Alternatively, the connection plug 51 may be ultrasonically welded to the inside of the port 3. The O-ring seal 25 is then securely joined to the connection plug 51, with the locking ring 39 sandwiched between the connection plug 51 and the O-ring seal 25 to form a tight fit between the components of the catheter connection assembly 15. Thus, in one exemplary aspect, the secondary portion of the connection plug 51 can be configured to be inserted therein a portion of the O-ring seal lumen 32 such that the inner back face 34 abuts the locking ring 39 and substantially fixes the position of the locking ring 39 between the O-ring seal 25 and the connection plug 51.

In another exemplary aspect, in which the circumferential edge 47 of the locking ring 39 is positioned in a locking ring plane substantially transverse to a longitudinal axis of the port bore 4, the inner back face 34 of the connection plug 51 can be positioned to abut the circumferential edge 47 of the locking ring 39. In this aspect the inner back face 34 extends substantially parallel to the locking ring plane such that a portion of the inner back face 34 of the connection plug 51 acts to prevent outwardly movement of the plurality of finger members 41 past the locking ring plane and outwardly toward the face or side wall 2 of the vascular access port 3.

The advantage of the catheter connector assembly 15 of the present invention is that a low insertion force is required to insert the catheter 11 into the vascular access port 3, compared to other catheters in the prior art. For example, as illustrated in Table 1 below, an average of 1.02 pounds of insertion force is required to insert the catheter 11 into the vascular access port 3 with the catheter connector assembly 15, which is considerably less compared to the 7.40 pounds of insertion force for the prior art catheter connection. In the second column, n is the number of trials that were performed. The average is the average number of pounds of insertion required to securely connect the catheter to the vascular access port.

TABLE 1

| | Port with Catheter Connector | | | | | | | | | |
|---|---|---|---|---|---|---|---|---|---|---|
| | Assembly | | | | | Prior art catheter connector | | | | |
| Test | n | Ave | Std | Hi | Lo | n | Ave | Std | Hi | Lo |
| Insertion | 20 | 1.0200 | .0638 | .9000 | 1.1000 | 5 | 7.4000 | 0.8065 | 8.7000 | 6.6000 |
| in lbs. | | | | | | | | | | |

The catheter connection of the current invention is also advantageous in that it provides superior catheter port securement. As shown below in Table 2, the amount of axial tensile force required to disconnect the catheter shaft 11 from the vascular access port 3 with the catheter connector assembly 15 was tested in dry and in wet conditions and compared with a prior art catheter connector.

The port bore 4 has an inner geometry that is adapted to house the catheter connector assembly 15 in such a manner as to allow clearance room for the finger members 41 to flex in one direction only. Particularly, the port bore 4 allows the finger members 41 of the locking ring 39 to flex distally toward the port reservoir 10 and proximally away from the longitudinal axis of the port bore 4. This allows a practitioner to use minimal insertion force to insert a catheter 11 into the port 3. Simultaneously, the finger members 41 are also prevented from moving in the opposite direction, i.e., toward the vascular access port opening 7.

TABLE 2

| | Port with catheter connector | | | | | |
|---|---|---|---|---|---|---|
| | assembly | | | Prior art catheter connector | | |
| | | | Std | | | Std |
| Test | Number | Average | Dev | Number | Average | Dev |
| Tensile test | 8 | 9.1000 lbs | .3436 | 10 | 3.4200 lbs | .6847 |
| Dry | | | | | | |
| Tensile test | 10 | 7.5400 lbs | .5959 | 10 | 3.0200 lbs | .1105 |
| Wet | | | | | | |

In dry conditions, for example, after 8 trials, the average amount of force required to disconnect the catheter 11 from the vascular access port 3 was 9.10 pounds. In wet conditions, such a practitioner might encounter in a surgical environment, after 10 trials the average amount of force required to release the catheter 11 from the port was 7.54 lbs. As can be seen from Table 2, the catheter connection of the present invention is also advantageous over prior art catheters because approximately 9 or 10 pounds of force is required to disconnect the catheter 11 from the port 3, as opposed to approximately 3 to 3.4 pounds of force of prior art catheter. This is desirable because it means that the connection between the catheter 11 and the port 3 is more secure compared to the catheter connection of the prior art catheter. Additionally, the catheter connector assembly 15 of the present invention is able to withstand high pressures, for instance, between 300 to 350 psi without leaking or otherwise compromising the port integrity.

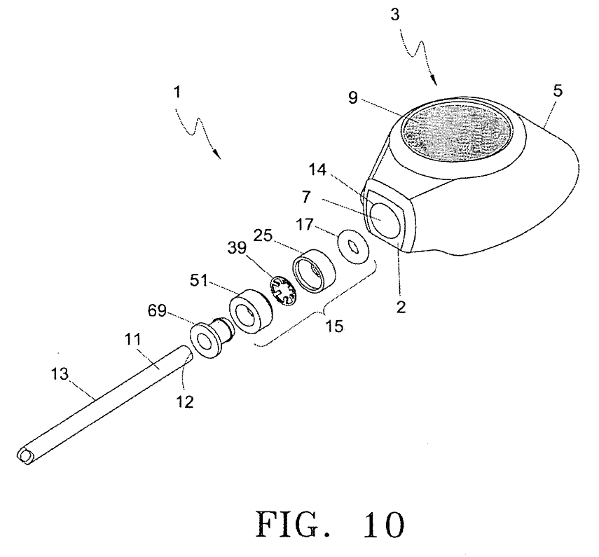

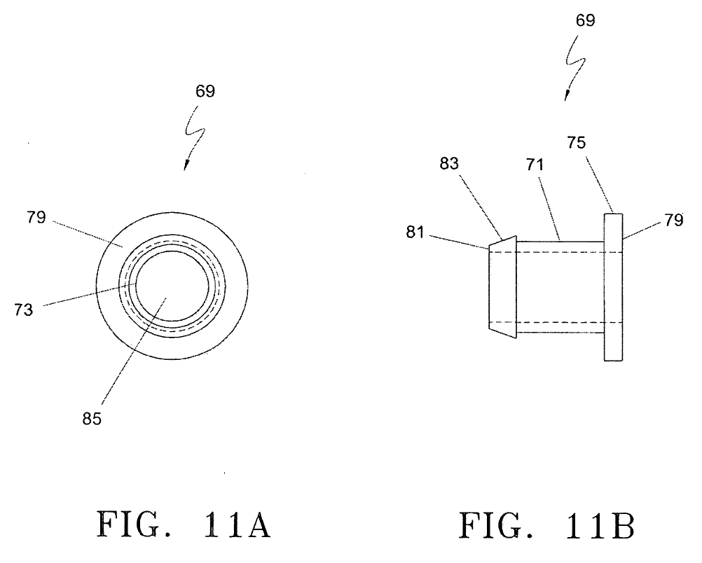

FIGS. 10 and 11 illustrate an additional embodiment of the catheter connector assembly 15 of the present invention in which a catheter release mechanism 69 may be used to disconnect the catheter 11 from the vascular access port 3. This feature is advantageous if the practitioner needs to replace the catheter or port due to complications while leaving the other component in place.

The catheter release 69 is positioned in a coaxial arrangement with the connection plug 51 and the catheter shaft 11. Referring to FIG. 11B, the catheter release 69 has a body 71 with an outer surface, a through lumen 85, an inner wall 73, a front face 79 with an outer rim 75, a barb 83, terminating in a back face 81. The front face 79 with an outer rim 75, form a flange, that is joined to barb 83 by the body 71 of the catheter release 69. The catheter release 69 has an outer diameter of approximately 0.250 inches at the outer rim 75 of the front surface 79 and an inner diameter of approximately 0.095 inches. The wall thickness of the front surface 79 of the catheter release 69 is approximately 0.030 inches. The overall length of the catheter connection release 69 is approximately 0.220 inches. The catheter release 69 may be composed of a hard plastic or engineering resin such as polysulfone or acetal, although any suitable material may be used.

In one embodiment the front face 79 and outer rim 75 of the catheter release 69 remains outside of the vascular access port bore 4, while the barb 83 and a portion of the body 71 of the catheter release 69 are inserted into of the vascular access port 3. Approximately 0.010 inches of the catheter release 69 body 71 remains outside of the port bore 4 after insertion. The body 71 of the catheter release 69 is inserted into the lumen 85 of the catheter connection plug 51 to form a tight fit. The catheter connection plug 51 and O-ring seal 25 in this embodiment have modified lumens with larger luminal diameters and inner profiles that conform to the outer contour of the catheter release 89. In the assembled state of the catheter connector assembly 15, the barb 83 of the catheter release 69 abuts up against portions of the front edges of locking ring finger members 41 of the locking ring 39 when the catheter release mechanism 68 is positioned in a first locking position.

In one aspect to release the catheter from the port, the front face 79 of the catheter release 69 is manually pushed inwardly along the longitudinal axis of the port bore 4 to the second unlocking position by approximately 0.010 inches. Thus, the barb 83 with leading edge 81 of the catheter release 69 is complementarily axially moved into position against the locking ring finger members 41 and forces the plurality of locking ring finger members 41 to move from their second, biased position, such as described above, in which the locking ring finger members 41 may be slightly embedded in the outer surface 13 of the catheter wall 11, inwardly and away from the longitudinal axis of the port bore 4. In the second, unlocking position, the locking ring lumen 49 has a diameter that is larger than the outer diameter of the catheter, which enables the outer wall 13 of the catheter shaft 11 to become free from the locking ring finger members 41 of the vascular access port 3, thereby releasing the catheter 11.

A method of connecting a catheter 11 with an implantable vascular access port 3 using the catheter connector assembly 15 of the present invention is also provided. The method involves providing the vascular access port assembly 1 of the present invention. The vascular access port 3 has a septum 9, a reservoir 10, a port bore 4, a port channel 8, and a catheter connector assembly 15. The catheter connector assembly 15 is comprised of an O-ring 17; an O-ring seal 25; a locking ring 39; and a catheter connection plug 51. The method further involves inserting the end of catheter 11 into the vascular access port bore 4, advancing the distal end 12 of the catheter 11 through the catheter connector assembly 15, consecutively through the catheter connection plug 51, the locking ring 39, O-ring seal 25, and the O-ring, until the catheter end abuts up against the channel of the vascular access port, and locking the locking ring 39 around the outer wall 13 of the catheter 11, thereby preventing the catheter from being released from the port 3. Optionally, the method may include releasing the catheter shaft 11 from the vascular access port 1 by engaging a catheter release mechanism 69 which flexes the finger members 41 of the locking ring 39 into a second, unlocking position thereby allowing the catheter to become released from the catheter connection assembly 15.

The above catheter connector assembly 15 thus solves the problems in the prior art by requiring a lower insertion force to insert the catheter 11 into the port 3. The catheter connector assembly 15 also provides a secure locking mechanism for the catheter 11 once it is inserted into the vascular access port 3, which does not compromise the lumen cross-sectional area, which thereby helps to maintain fluid communication between the port 3 and the lumen of the catheter 11. The catheter connection assembly 15 also prevents problems associated with having to align a catheter with a stem located outside of the port 3 or advancing a catheter 11 over a barbed stem design. The foregoing proposed catheter connector assembly could be applied to many different types of vascular access ports.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g., each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes description of the preferred and alternate embodiments of the invention. Those skilled in the art may recognize other equivalents to the specific embodiment described herein which equivalents are intended to be encompassed by the claims attached hereto.

What is claimed is:

1. A catheter connection assembly for connecting a catheter having an outer wall that has an outer diameter with a vascular access port that defines a port bore, wherein the port bore is adapted to house the catheter connection assembly within a portion of the port bore, and a channel that is in fluid communication with the port bore and a reservoir, the catheter connection assembly comprising:
a locking ring configured to be positioned therein the port bore, the locking ring comprising a plurality of finger members that extend inwardly from a circumferential edge of the locking ring, each finger having an inner surface, wherein the respective inner surfaces of the plurality of fingers define a locking ring lumen, in which a portion of the outer wall of the catheter is positioned therein the locking ring lumen in frictional contact with the finger members, wherein the finger members are configured to flex from a first, unbiased, position to a second, biased, position, when the outer wall of the catheter is positioned within the locking ring lumen.

2. The catheter connection assembly of claim 1, wherein the locking ring lumen has a first diameter that is less than the outer diameter of the catheter when the finger members are in the first, unbiased position, and wherein the locking ring lumen has a second diameter that is equal to or greater than the first diameter and is less than or substantially equal to the outer diameter of the catheter when the plurality of finger members is in the second, biased position.

3. The catheter connection assembly of claim 1, wherein the locking ring comprises a shape-memory alloy.

4. The catheter connection assembly of claim 1, wherein the finger members are configured to flex unidirectionally, upon positioning of the outer wall of the catheter within the locking ring lumen in frictional contact with the finger members.

5. The catheter connection assembly of claim 4, wherein the finger members are configured to flex inwardly toward the direction of the port reservoir.

6. The catheter connection assembly of claim 1, wherein the plurality of finger members are angularly spaced from each other at substantially the same distance so that force exerted on the outer wall of the catheter by the finger members can be substantially equalized.

7. The catheter connection assembly of claim 1, wherein the circumferential edge of the locking ring is positioned in a locking ring plane substantially transverse to a longitudinal axis of the port bore, and wherein, in the first, unbiased, position, the plurality of finger members extends inwardly away from the locking ring plane toward the port reservoir at an acute angle relative to the longitudinal axis of the port bore.

8. The catheter connection assembly of claim 1, further comprising a means for fixedly positioning the locking ring relative to a portion of the port bore.

9. The catheter connection assembly of claim 1, further comprising a means for fluidically sealing against the outer wall of the catheter and a portion of the port bore to prevent vascular access port leakage via the port bore.

10. The catheter connection assembly of claim 1, wherein the port bore comprises a first portion that extends inwardly from a side wall of the vascular access port to a shoulder surface and a second portion that extends inwardly from the shoulder surface and terminates at an edge of the channel, wherein the first portion has a diameter that is greater than the diameter of the second portion, and wherein the channel has a diameter that is less than the outer diameter of the catheter.

11. The catheter connection assembly of claim 10, further comprising an O-ring configured to be seated against the shoulder surface of the port bore and defining an O-ring lumen that has a diameter that is less than the outer diameter of the catheter, wherein the O-ring lumen is configured for an interference fit with the outer wall of the catheter.

12. The catheter connection assembly of claim 11, further comprising an O-ring seal having an outer wall, an inner surface, a front facing outer surface, and an inwardly facing outer surface, the inner surface defining an inwardly tapering O-ring seal lumen, wherein the diameter of the outer wall of the O-ring seal is substantially the same as the diameter of the first portion of the port bore and is configured to be received therein a portion of the first portion of the port bore, and wherein the inwardly facing outer surface abuts the O-ring such that the O-ring lumen and the O-ring seal lumen are substantially co-axial.

13. The catheter connection assembly of claim 12, wherein the locking ring is mounted therein a portion of the inner surface of the O-ring seal, proximate to the front facing outer surface of the O-ring seal.

14. The catheter connector assembly of claim 13, further comprising a connection plug having a primary portion and a secondary portion of reduced diameter, wherein the diameter of the primary portion of the connection plug is substantially the same as the diameter of the first portion of the port bore and is configured to be received therein a portion of the first portion of the port bore, wherein the secondary portion has an inner back face and is configured to be inserted therein a portion of the O-ring seal lumen such that the inner back face abuts the locking ring and substantially fixes the position of the locking ring between the O-ring seal and the connection plug.

15. The catheter connection assembly of claim 14, wherein the circumferential edge of the locking ring is positioned in a locking ring plane substantially transverse to a longitudinal axis of the port bore, and wherein the inner back face of the connection plug abuts the circumferential edge of the locking ring and extends substantially parallel to the locking ring plane such that a portion of the inner back face of the connection plug acts to prevent outwardly movement of the plurality of fingers past the locking ring plane and toward the side wall of the vascular access port.

16. The catheter connection assembly of claim 14, wherein the connection plug defines a connection plug lumen, and wherein the locking ring lumen, the O-ring lumen, the O-ring seal lumen, and the connection plug lumen are substantially co-axial.

17. The catheter connection assembly of claim 16, wherein the catheter is configured to be received therethrough the connection plug lumen, the locking ring lumen, the O-ring seal lumen, and the O-ring lumen with a distal end of the catheter extending beyond the O-ring and into contact with the edge of the channel.

18. The catheter connection assembly of claim 16, further comprising a catheter release mechanism comprising a body defining a lumen extending therethrough that is configured to slideably receive the catheter, wherein a proximal portion of the catheter release mechanism comprises a flange and an opposed distal portion of the catheter release mechanism comprises a barb, wherein the catheter release mechanism is configured to be operatively positioned therethrough the connection plug lumen, and wherein, in a first, locking position, the barb of the catheter release mechanism is positioned abutting portions of the plurality of fingers of the locking ring, and the flange is spaced from an outer front surface of the connection plug.

19. The catheter connection assembly of claim 18, wherein the catheter release mechanism is configured to be moved axially along the longitudinal axis of the port bore to a second, unlocking position, wherein the flange is moved axially toward the outer front surface of the connection plug such that the barb is complementarily axially moved which forces the plurality of finger members to be moved inwardly and away from the longitudinal axis of the port bore in which the locking ring lumen has a diameter that is larger than the outer diameter of the catheter.

20. A catheter connection assembly for connecting a catheter having an outer wall that has an outer diameter with a vascular access port that defines a port bore and a channel that is in fluid communication with the port bore and a reservoir, wherein the port bore has a first portion that extends inwardly from a side wall of the vascular access port to a shoulder surface and a second portion that extends inwardly from the shoulder surface and terminates at an edge of the channel, wherein the first portion has a diameter that is greater than the diameter of the second portion, the catheter connection assembly comprising:
a locking ring configured to be positioned: therein the port bore, the locking ring comprising a plurality of finger members that extend inwardly from a circumferential edge of the locking ring, each finger having an inner surface, wherein the respective inner surfaces of the plurality of fingers define a locking ring lumen, in which a portion of the outer wall of the catheter is positioned therein the locking ring lumen in frictional contact with the finger members, wherein the finger members are configured to flex from a first, unbiased, position to a second, biased, position, when the outer wall of the catheter is positioned within the locking ring lumen;
an O-ring configured to be seated against a shoulder surface of the port bore and defining an O-ring lumen that has a diameter that is less than the outer diameter of the catheter, wherein the O-ring lumen is configured for an interference fit with the outer wall of the catheter; and
an O-ring seal having an outer wall, an inner surface, a front facing outer surface, and an inwardly facing outer surface, the inner surface defining an inwardly tapering O-ring seal lumen, wherein the diameter of the outer wall of the O-ring seal is substantially the same as the diameter of the first portion of the port bore and is configured to be received therein a portion of the first portion of the port bore, and wherein the inwardly facing outer surface abuts the O-ring such that the O-ring lumen and the O-ring seal lumen are substantially co-axial.

21. The catheter connection assembly of claim 20, further comprising a connection plug having a primary portion and a secondary portion of reduced diameter and a connection plug lumen, wherein the diameter of the primary portion of the connection plug is substantially the same as the diameter of the first portion of the port bore and is configured to be received therein a portion of the first portion of the port bore, wherein the secondary portion has an inner back face and is configured to be inserted therein a portion of the O-ring seal lumen such that the inner back face abuts the locking ring and substantially fixes the position of the locking ring between the O-ring seal and the connection plug.

22. The catheter connection assembly of claim 20, further comprising a catheter release mechanism comprising a body defining a lumen extending therethrough that is configured to slideably receive the catheter, wherein a proximal portion of the catheter release mechanism comprises a flange and an opposed distal portion of the catheter release mechanism comprises a barb, wherein the catheter release mechanism is configured to be operatively positioned therethrough the connection plug lumen, and wherein, in a first, locking position, the barb of the catheter release mechanism is positioned abutting portions of the plurality of fingers of the locking ring and the flange is spaced from an outer front surface of the connection plug.

23. A method of inserting a catheter into a vascular access port, wherein the vascular access port defines a port bore and a channel that is in fluid communication with the port bore and a reservoir, the port bore terminating at an edge of the channel, wherein the catheter has an outer wall that has a catheter diameter, the method comprising:
positioning a locking ring therein the port bore substantially transverse to a longitudinal axis of the port bore, wherein the locking ring comprises a circumferential edge and a plurality of fingers, each finger having an inner surface that extends inwardly from the circumferential edge, and wherein the respective inner surfaces of the plurality of fingers define a locking ring lumen having a first diameter that is less than the outer diameter of the catheter when the fingers are in a first, unbiased position; and
positioning the catheter therein the port bore and therethrough the locking ring lumen, wherein a portion of the outer wall of the catheter comes in frictional contact with the respective inner surfaces of the plurality of fingers and forces the fingers from the first, unbiased position, to a second, biased position in which the locking ring lumen has a second diameter that is substantially equal to or greater than the first diameter and is less than or substantially equal to the outer diameter of the catheter.

24. The method of claim 23, further comprising fixedly seating the locking ring within a portion of the port bore.

25. The method of claim 23, further comprising sealing a portion of the port bore about the outer wall of the inserted catheter so that there is no port leakage via the port bore.

26. The method of claim 23, further comprising:
positioning a catheter release mechanism such that a portion of the mechanism abuts portions of the plurality of fingers of the locking ring; and
selectively moving the catheter release mechanism axially along the longitudinal axis of the port bore toward the channel to force the plurality of finger members to be moved inwardly toward the channel and away from the longitudinal axis of the port bore such that the locking ring lumen expands to a diameter that is larger than the outer diameter of the catheter, thereby allowing the catheter to be removed from the vascular access port.
